(19) United States
(12) Patent Application Publication
(10) Pub. No.: US 2002/0056147 A1
(43) Pub. Date: May 9, 2002

(54) Transgenic identification markers

(76) Inventors: Peter C. Dau, Winnetka, IL (US); Debang Liu, Wilmette, IL (US)

Correspondence Address: McGuireWoods, LLP, Suite 1800, 1750 Tysons Boulevard, McLean, VA, 22102, US

(21) Appl. No.: 09/916,203

(22) Filed: Jul. 27, 2001

Related U.S. Application Data
(60) Provisional application No. 60/221,520, filed on Jul. 28, 2000.

(51) Int. Cl. A01K 67/00; A01H 5/00; C12Q 1/70; C12Q 1/68; C12N 7/01; C12N 1/21

(52) U.S. Cl. 800/8; 800/278; 435/6; 435/5; 435/252.3; 435/235.1

(57) ABSTRACT

The present invention provides transgenic identification markers (TIMs) and methods of their use for identifying organisms and their progeny. TIMs are synthetically produced, heritable DNA molecules that, when inserted into cells of an individual organism, constitute a distinguishing, synthetic marker system for the organism. Because TIMs are heritable, upon cell division they are passed on to the progeny of the marked organism. TIMs thus provide a means of identifying and distinguishing such marked organisms and their progeny.

This application claims the benefit of United States Provisional Application 60/221520 filed on Jul. 28, 2000 under 35 USC 119(e) and the complete contents of that application is herein incorporated by reference.

### DESCRIPTION

### BACKGROUND OF THE INVENTION

1. Field of the Invention

The invention generally relates to the detection of genetically modified organisms (GMOs). In particular, the invention provides DNA sequences denominated Transgenic Identification Markers (TIMs) for use in the detection of genetically modified cells or organisms in which DNA is the heritable genetic material.

2. Background of the Invention

Due to advances in recombinant DNA technology genetically modified organisms (GMOs) are becoming increasingly prevalent. Most commonly transgenic, but also isogenic alterations are being undertaken (Moffat, 2000). A leading area of application of this new technology has been in crop science with the goal of enhancing the nutrition of a rapidly expanding global human population (Kishore, 1999); another has been the development of transgenic animals (Miesfield, 1999). It is estimated that by 2020 global demand for the major cereal crops, rice, wheat and maize, will increase by 40% (Mann, 1999a). It is not possible to increase food productivity this much in such a short period of time by conventional methods of breeding and selection (Vasil, 1999).

In North America alone, almost 20×10⁶ ha of GMO plants were planted in 1999. 2 5 The vast majority of these crops are the result of single-gene transfers, in which one or more genes coding for desired traits such as herbicide (Mann) or pathogen resistance (Gao, 2000) are transformed into the genome of the crop plant from an outside source. Forty-six such products had been received by the FDA by the year 2000 for pre-marketing approval (FDA, Office of Premarket Approval, 1999) and it is estimated that over 7000 distinctive transgenic plants have been engineered. In order to substantially increase food production even more extensive plant engineering will likely have to be undertaken in the future involving entire metabolic pathways such as photosynthesis (Mann, 1999b). In addition to their nutritional uses plants are also being modified for exotic traits such as edible vaccine production (ProdiGene), antibody production (Integrated Protein Technologies), and plastics (Cargill Dow Polymers).

Based upon the increasing stream of GMOs there is an increased need for simple and reliable means of identification of transgenic and isogenic plants and livestock, their progeny, and the products derived from them. To mention a few problems, some foreign countries want verifiable exclusion of transgenic products from imported crops. Organic farmers and producers need to be able to exclude transgenic products from the foods they market. The horizontal transfer of genes via cross pollination between transgenic and natural plants (weeds) needs to be monitored. Seed companies need to be able to detect unauthorized use of their seeds by producers. There needs to be a quick and reliable method for researchers to identify the multiple GMOs produced in the research facility and tested in field trials. Finally, transgenic or cloned livestock and their products will need to be simply and reliably differentiated from each other. In response to these needs and concerns the FDA is planning to issue guidelines for voluntary labeling. To ensure that the labeling meets Federal Food, Drug, and Cosmetic Act (FFDCA) standards, the U.S. Department of Agriculture is developing a program to certify laboratories and testing kits for the detection of bioengineered components of food (Goldman, 2000).

Testing for a GMO trait could be a straightforward assay for a DNA insert or its RNA or protein product. Isogenic traits might be similar to the native states and accordingly might be more difficult to identify. The size and location of a DNA insert and the location of suitable PCR binding sites have to be considered for an assay utilizing PCR amplification and identifying the products by electrophoresis or by hybridization. PCR products might not amplify well because of excessive length, low concentration, degradation of the target sequence or secondary DNA structure. Protein products might be poorly detectable because of low concentration, degradation or a technically inadequate reporter system. Further, multiple insertions of identical or similar genes need to be distinguishable.

A common method to determine whether foodstuffs contain genetically modified ingredients involves purification of genomic DNA from the material in question and subsequent analysis by polymerase chain reaction PCR® amplification. Two of the most important factors directing transgene expression in plants are the promoter and terminator elements used to control transcription of the foreign gene. Two of the most common genetic elements employed in transgene expression are the constitutive 35S promoter from the cauliflower mosaic virus (CaMV) and the 3′ terminator isolated from the nopaline synthase gene (NOS) of Agrobacterium tumefaciens. These two genetic elements are often targeted in PCR-based GMO tests, as they do not naturally occur in agricultural crop sources, are widely present in commercial GMO-containing foods and can be detected with great sensitivity (U.S. Pat. No. 6,027,945). However, this method does not distinguish between different GMOs employing these promoter or terminator elements.

Yoder et al. (International Publication # WO 92/01370) have disclosed a method for identifying the progeny of a plant through creating a molecular fingerprint in the genome of the plant by inserting a DNA fingerprinting construct into the genome. The construct contains a transposon or other foreign DNA. Insertion of the construct into the host DNA creates a restriction length polymorphism that can be used to detect a plant or its progeny. However, the insertion of the construct is random. Therefore, the use of this method risks insertion of the construct into essential areas of the genome and potential loss of the function of those areas, which could be detrimental to the plant and/or its progeny. Further, the technique is not designed to take advantage of PCR amplification technology but instead relies on Southern blot analysis of the restriction length polymorphisms which are generated.

One approach to the analysis of naturally occurring genetic variation, and thus of genetic identification, is the analysis of short tandem repeat (STR) loci. STR loci have been found to be extremely useful as genetic markers. STR repeat DNA sequences are 2-7 bp in length and are found throughout the plant and animal kingdoms (Bowling, 1997; Sanchez-Escribano, 1999; Swanston, 1999; Tessier, 1997; Waldbieser, 1997; Weikard, 1997; and Yu, 1999). These loci are highly polymorphic with respect to the number of repeat units they contain and may vary in internal structure as well. Variation in the number of STR repeat units at a particular locus causes an identifiable length variation of the DNA at that locus among different populations within a species or different species. Many allelic STR variants may exist within a population, providing a rich source of easily scored genetic variation.

Naturally recurring tandem repeat loci from a number of sources have been extensively characterized and have been found to vary in the sequence of their repeat units. In a survey of (CA)n or (GT)n human STR loci 64% were perfect simple repeats without interruption, 25% were imperfect repeat sequences with one or more interruptions in the run of repeats and 11% were compound repeat sequences with adjacent simple repeats of a different sequence (Weber, 1990). A survey of plant loci ranging from mono- to tetranucleotide repeated sequences in the EMBL and GenBank nuclear DNA sequence databases showed (AT)n sequences to be the most abundant followed by (A)ₙ·(T)ₙ, (AG)ₙ·(CT)ₙ, (AAT)ₙ·(ATT)ₙ, (AAC)ₙ·(GTT)ₙ, (AGC)ₙ·(GCT)ₙ, (AAG)ₙ·(CTT)ₙ, (AATT)ₙ·(TTAA)ₙ, (AAAT)ₙ·(ATTT)ₙ, and (AC)ₙ·(GT)ₙ. There was 1 repeat locus in every 23.3 kb of DNA (Wang, 1994).

The incidence of perfect STRs of >12 bp including (A)ₙ·(T)ₙ tracts was reported by Toth, 2000, across 8 taxa. (A)ₙ·(T)ₙ, tracts were more abundant in each taxon than (C)ₙ·(G)ₙ tracts, which were rare. Mononucleotide repeats were replaced by the dinucleotide AT repeat as the most frequent type in primate, plant and yeast intergenic regions. AC and AG were intermediate. In vertebrates and arthropods, AC was the most frequent dinucleotide STR. In C. elegans it was AG. Among trinucleotide STRs, AAC and AAG were most frequent in plant exons. Generally, there was a lack of ACG and ACT trinucleotide STR in most taxa, however CAG was frequent. In all vertebrates (G+C)-rich repeats dominated in exons, whereas they were less numerous in introns and intergenic regions.

CCG repeats were quite significant in all vertebrates except in introns, where they may be selected against because of the requirements of the splicing machinery. They are under represented in other taxa. The absence of CCG and ACG repeats from introns of all vertebrates could be explained by the presence of the highly mutable CpG dinucleotide within the motif because intergenic CpG regions may remain unmethylated in vertebrates. The very low frequency of ACT trinucleotide repeats in all sequences was striking. It cannot be explained by the presence of a stop codon on one strand since genomic regions other than exons were also affected. Intergenic regions of arthropods and vascular plants showed an excess of AAC and AAG repeats.

Tetranucleotide repeats represented a higher proportion of all vertebrate genomes than triple repeats, in spite of the fact that exons seem to tolerate only trinucleotide and hexanucleotide repeats effectively. Tetranucleotide STR with <50% G+C were most abundant with the notable exception of AAGG. For all taxa the most frequent tetranucleotide repeats were AAAT, AAAC, AAAG, AAGG, AGAT, ACAG, ACAT and ACCT.

Microsatellite loci undergo mutation usually involving the gain or loss of single, entire repeat units, most commonly the former (Primmer, 1996). In the study of a hypervariable swallow tetranucleotide repeat locus, 26 gains and 7 losses of repeat units were observed. Only 6 changes involved gain or loss of more than one unit. Mutation events were biased towards longer units, which may be a manifestation of the same mechanism underlying the correlation between repeat length and polymorphism of individual loci (Weber, 1990).

A gain over loss of repeat units was also recently reported for a dinucleotide repeat unit in a tissue culture system (Vergunst, 1999). A tetranucleotide STR locus within human pedigrees varied by gain or loss of exactly 1 repeat unit relative to that of the original parental chromosome (Mahtani, 1993). Their finding of complete linkage disequilibrium between each of 2 closely flanking insertion/deletion polymorphisms suggested that the new mutant alleles of the tetranucleotide repeat likely arise by polymerase slippage or unequal sister chromatid exchange and not by unequal exchange between alleles.

The primary mutational mechanism leading to changes in microsatellite length is polymerase template slippage (Schlotterer, 1992; Strand, 1993). During replication of a repetitive region, DNA strands may dissociate and then reassociate incorrectly. Renewed replication in the misaligned state leads to insertion or deletion of repeat units, thus altering allele length. In microsatellite loci where rapid growth does not occur, most of the observed changes in length are +1 repeat. An upper bound on the number of repeat units in microsatellites is based on the observation that very long alleles are rare (Goldstein, 1997).

An explanation for the absence of very long alleles is that point mutations within a repeat unit interrupt the microsatellite repeat region creating two shorter repeat regions (Schug, 1998; Vosman, 1997).

Characterization of alleles at specific STR loci for purposes of individual identification usually begins with their PCR amplification from genomic DNA of the individual organism whose genome contains those loci. Although a particular repeat unit may be common to several different STR loci, identification of a particular STR locus is effected by PCR amplification with primer pairs hybridizing to unique DNA sequences which flank the repeat region, i.e. unique sequences located 5′ and 3′ to a central repeat region. Use of unique flanking sequence primers makes it possible to simultaneously amplify many different STR loci in a single DNA sample, a technique referred to as multiplexing. The resulting PCR products (amplicons) from the various loci may then be separated by electrophoresis and identified by determining their individual lengths in comparison to known DNA standards. Alternatively, PCR amplicons for STR loci are analyzed by mass spectrometry or hybridization technologies.

It would be of highly beneficial to have available a method of identifying GMOs that possesses the attributes of STR analysis, i.e. that provides highly diverse, stable, heritable genetic markers for GMOs that can be readily assessed. Such a system could take advantage of the extensive knowledge and technology that has developed around STR analysis, and utilize that knowledge and technology for the identification of GMOs and their progeny.

### SUMMARY OF THE INVENTION

It is an object of the present invention to provide Transgenic Identification Markers (TIMs) for the purpose of identifying genetically modified organisms and their progeny. TIMs are DNA molecules which can be chemically synthesized or produced by recombinant DNA technologies including the polymerase chain reaction (PCR) employing artificial DNA templates.

They contain an identifying central region (ICR) bounded by 5′ and 3′ specific flanking regions (FRs). The specific FRs are designed to contain 5′ and 3′ primer binding sites relative to the ICR which are not sufficiently homologous to native DNA in the cell or organism to allow its amplification by PCR. Alternatively, the primer binding sites are located at distal sites within the native DNA such that PCR amplification of the host DNA using homologous primers will not produce a PCR amplified product. TIMs may contain restriction enzyme sites in order to allow ligation detection of ICR. TIMs may further contain differential primer binding sites to allow independent amplification of multiple ICR within the same genome.

The ICR may be composed of (1) one or more tandemly repeated sequences of 2-7 bp which may contain interspersed extra nucleotides and vary in length by their number of repeat units or interspersed nucleotides. Repeated sequences will usually be identical, but may be heterogenous. ICR may alternatively be comprised of (2) non-repeated DNA sequences or 3) mixtures of 1 and 2..

Tandemly repeated ICR, as well as some non-repeated sequences or mixed ICR sequences, may vary in fragment length and mass so that they can be differentiated from one another by an appropriate detection system, e.g., electrophoresis or mass spectrometry. Non-repeated ICR sequences of the same length will vary in sequence so that they can be differentiated from one another by a sequence sensitive detection method such as DNA sequencing or chip hybridization. Tandemly repeated ICR could also be detected by these methods.

Together with their restriction sites and specific flanking sequences TIMs can be inserted by the appropriate gene transfer technologies into entities to be identified. They can be inserted into the host entity either covalently linked to or separate from other transferred DNA sequences designed to create a genetic modification of the entity. An entity need not be otherwise genetically modified to be genetically marked by a TIM. Multiple covalently linked TIMs (FIG. 1), individual TIMs or multiple individual TIMs can be transferred. Once within the host entity, the TIMs may be maintained either by integration into the host genome, or extrachromasomally.

TIMs are “heritable” in that they are replicated in the host entity during organelle or cellular replication and then passed on to the daughter cells. They also appear in the germ cells of organisms so that they are transmitted to progeny by sexual reproduction.

TIMs can be retrieved from the target cells or organisms by PCR amplification with one or more primers hybridizing in their specific flanking sequences, and identified by measurement of their fragment length, mass or sequence upon application of the requisite analytical technology.

One or more TIMs are inserted into organelles or cells in culture or of an individual organism to form a synthetic marker system useful in identifying those organelles or cells and their progeny and discriminate that individual and its progeny from other cells or organisms. Identical 5′ and 3′ flanking sequences providing primer binding sites for the amplification of ICR can be employed with a large number of different ICR, thereby providing a means to screen for many different ICR with the same PCR reaction. A new TIM database for each pair of 5′ and 3′ flanking sequences providing primer binding sites could be created from an existing ICR database. A new database could therefore be created for different cells or organisms employing the same ICR by changing the 5′ and/or 3′ flanking sequences to hybridize with one or more different PCR primers which did not hybridize sufficiently with the original flanking sequences to allow PCR amplification. A TIM from an unknown cell or organism would become known after PCR amplification by application of the relevant analytical technology and matched to the database for its identification and association with physiological DNA transformants.

Simple pyrimidine repeats such as aaat, or pyrimidine/purine repeats, such as atag, could be employed as TIM repeat units. By avoiding predominantly purine tracts, TIMs can be designed to have a low degree of secondary structure to avoid interference with their PCR amplification. The size and number of repeat units and their interspersion with other repeat units or nucleotides would be selected to permit easy detection and discrimination from other closely related TIMs by the analytical instrumentation to be employed. A regular repeat unit structure would not have to be maintained, depending upon the desired level of analytical resolution required.

Di-nucleotide repeat units may show “shadow band” amplification artifacts caused by DNA polymerase slippage during PCR (Hauge, 1993). Therefore, repeat units of 3-5 bp are often preferred in genetic analysis. Some trimeric repeats may undergo expansion within an organism possibly due to the formation of unusual secondary structures during replication. Their expansion may be blocked by interruptions within the repeated DNA (Samadashwily, 1997). Longer microsatellite repeat units of 6 and 7 nts have the disadvantage of making the TIM longer for a given number of repeats thereby lessening the variety of detectable ICR within a given length of nucleotide sequence. A specific primer pair designed to amplify a TIM would be screened for non-specific amplification of the target genome. Although there may be some sequence homology between a PCR primer pair and the host genome, it is very unlikely that homologous sequences would be located close enough together in the correct orientation to allow for PCR amplification by a given primer pair designed for TIM amplification.

Most preferably, tetrameric repeat units would be selected. Their informativeness could be increased by interspersing single nt and di- and tri-nucleotide repeats. The least number of repeat units would be one, and the greatest number would be limited by the amplification, detection and gene transfer systems. For example, to obtain complete PCR amplification and rapid, accurate electrophoretic analysis of microsatellite loci, an upper limit of about 400 bp is frequently selected. In order to increase their power of discrimination, it is preferable to transfer multiple shorter TIMs rather than a single long TIM, since the power of the former increase geometrically, the latter arithmetically. Sets of TIMs of the same ICR length distinguishable by sequencing or hybridization could be constructed by use of different repeat units or interspersed nucleotides. This maneuver could be employed to increase the number of ICR available to label large numbers of cells or organisms, such as in distinguishing groups of organisms with transgenic modifications from groups of similar organisms with the same or similar physiological modifications produced at a different time, or with a different genetic modification.

In addition to regular tandem repeat sequences, non-repeat nucleotides can be introduced to cause the amplified fragment lengths from a TIM to vary in length by as little as a single nucleotide. For example, addition of 1, 2, or 3 nucleotides to 9 tetrameric repeats comprising 36 nt would create fragments of 37, 38, and 39 nt or repeats of 9.1, 9.2, and 9.3 units, thereby greatly increasing the discriminating power of a given length of sequence. The resolution of the analytical instrumentation would dictate whether such fractional repeat units could be discerned. For example, most sequencing gel electrophoresis systems can reliably discriminate DNA fragments differing by 2 bp; time of flight nuclear magnetic resonance can ordinarily discriminate a difference of 1 bp.

Since these TIMs will be unique by sequence, any variation in ICR sequence detectable by an analytical instrument would serve to discriminate different cells or organisms. The number of distinctive ICRs available for labeling could also be increased by changing their length. In the case of hybridization based detection instruments, the length of complementary DNA must be sufficient to provide enough bonding energy for specific hybridization (Wallace, 1997). Sequence variation in TIMs would be generally detectable by PCR followed by direct sequencing unless there were excessive secondary structure in the PCR amplicon.

TIMs are wholly or partially synthesized chemically or by recombinant DNA technology including PCR amplification. For example, chemically synthesized fragments with Eam 1104 I restriction sites at their 3′ and 5′ ends could be subjected to Eam 1104 I digestion and combined by T₄ ligation to produce fragments containing the desired central region. Flanking sequence deoxyribonucleotides could be synthesized 5′ and/or 3′ to the central region and contain a restriction site to allow their joining to other TIMs or cloning into a gene transfer vector. They would also incorporate primer binding sites designed to allow retrieval of the ICR from DNA of the target organism by PCR. Multiple unique 5′ and/or 3′ flanking sequences [intermediate flanking regions (IFR) (FIG. 1)] can be designed for multiple ICRs inserted tandemly into an organism. IFR could contain restriction sites to permit separation of the TIMs by restriction endonuclease digestion prior to a multiplex secondary PCR reaction. Alternatively, IFR could be designed to provide multiplex priming of TIMs without need for prior restriction digestion.

TIMs may be used as identification markers in any cell or organism receptive to transgenic modification in whom DNA is the heritable genetic material. Examples of such entities which can be a host for TIMs include but are not limited to: plants and cells or subcellular components derived from plants; animals and cells or subcellular components derived from animals; transgenic, isogenic, or chimeric organisms or cells; fungi; bacteria; viruses; insects; algae; protozoans; and the like. By “derived from” we mean, cells or subcellular components that are isolated from a multicellular organism or that have been propagated from other cells that were originally isolated from a multicellular organism. Further, TIMs may be transmitted to, propagated in and detected at any stage or form of the life cycle of such an entity.

It is a further object of the present invention to provide a host stably transfected with a artificial heritable transgenic identification marker. By “artificial” we mean that the heritable transgenic identification marker (i.e. the combination of the ICRs and flanking regions and internal flanking regions as described herein) does not occur naturally in the host into which it is inserted.

### BRIEF DESCRIPTION OF THE DRAWINGS

FIG. 1. Schematic outline of a TIM construct. ICR=identifying central region; FR=flanking region; IFR=intermediate flanking region; MRE=multiple restriction enzyme cutting sites.

FIG. 2. Schematic outline of a strategy for the detection of the ICRs of a multiple-ICR TIM. ICR=identifying central region; FR=flanking region; IFR=intermediate flanking region; 5′F=5′ forward primer; 3′R=3′ reverse primer. Circles represent labels.

FIG. 3. Schematic outline of a strategy for the detection of the ICR of a single-ICR TIM. ICR=identifying central region; FR=flanking region; 5′F=5′ forward primer; 3′R=3′ reverse primer. Circle represents label.

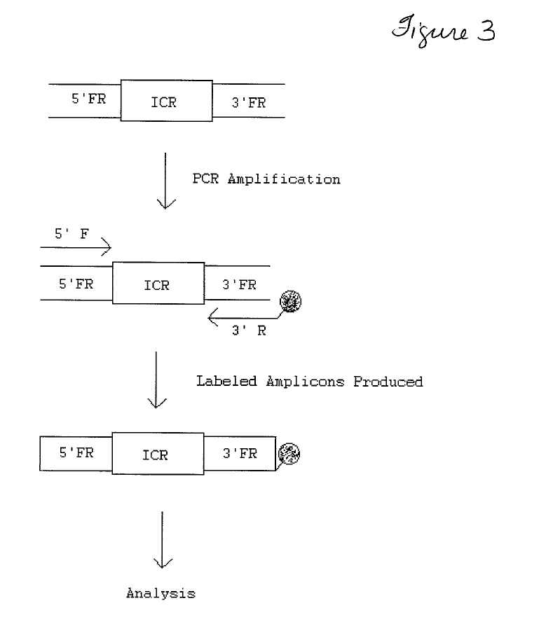

### DETAILED DESCRIPTION OF THE PREFERRED EMBODIMENTS OF THE INVENTION

Use of Transgenic identification markers.

The present invention provides methods for the use of Transgenic identification markers (TIMs). TIMs are heritable, synthetic DNA molecules which are inserted into the germ line cells of an entity for the purpose of genetically “marking” the entity and its progeny. Once an entity is marked with a TIM, it will be possible to identify and distinguish the entity and its progeny from, for example, otherwise similar or identical unmarked entities, and from entities marked with a different TIM.. TIM technology is thus applicable for monitoring and tracing, for example, genetically modified crops and animals, and products derived from such genetically modified entities.

In a preferred embodiment of the instant invention, the entity is an entity that has been genetically modified. However, those of skill in the art will recognize that it is not essential that the entities so marked and identified be genetically modified. The methods of the instant invention may also be utilized to mark and identify entities that are not genetically modified other than by insertion of the TIMs as described herein. For example, TIMs may be utilized as an internal genetic “tag” for, for example, purebred livestock, hybrid ornamental or crop plants, cultured cell lines, and the like, which have not been genetically modified.

TIMs may be used to mark an entity for any desired or useful reason. Reasons for the utilization of TIMs include but are not limited to the identification of: genetic modifications, institution of origin of the entity, year of development, and the like. Likewise, any type of entity may be marked for future identification of the entity itself, and/or for tracking and identification of its progeny. It is merely necessary that the entity to be marked with TIMs be amenable to insertion and retention of the TIMs. If the entity that originally receives the TIMs is the germ line cell of an organism destined to develop into a multicellular organism (as will generally be the case for multicellular organisms), the TIMs must be replicated and passed on to daughter cells during cellular division. The TIMs will thus be present and detectable in all cells of the mature, multicellular organism in which DNA is normally present. If the progeny of the entity are to be marked, it is also necessary that the TIMs be passed to the progeny of the entity during reproduction, and that the TIMs ultimately be present and detectable in most or all cells of the progeny at maturity.

In a preferred embodiment, the present invention provides a method of identifying genetically modified entities and their progeny. In a preferred embodiment of the instant invention, the genetically modified entity is a genetically modified organism (GMO). However, those of skill in the art will recognize that the method of the present invention can be utilized to identify genetically modified entities that are not, in a strict sense, organisms. Thus, the term “genetically modified entities” includes but is not limited to genetically modified cells (e.g. cultured cell lines, ex vivo cells which are intended for reimplantation into an organism, stem cells, differentiated cell lines to be used for a specific purpose e.g. neuronal replacement, and the like), subcellular organelles such as mitochondria, chloroplasts, plasmids, episomes, and the like.

The term “genetically modified entities” also encompasses “genetically modified organisms” or “GMOs”. The term “genetically modified organisms” refers to plants and animals containing genes transferred from other species to produce certain characteristics, such as resistance to certain pests and herbicides. Examples of GMOs that can be genetically identified by the methods of the instant invention include but are not limited to plants, animals, insects, fungi, viruses, and the like. Any organism, or cellular or subcellular component of an organism that is receptive to genetic modification, (i.e. into which TIMs may be inserted and retained) may be marked by the method of the instant invention. Further, the entities so marked may also be chimeric, transgenic or cloned.

In a preferred embodiment of the instant invention, the methods of the instant invention may be used for identifying both the originally marked entity and the progeny of that entity. Because TIMs are heritable, they are passed to the progeny of the organisms, cells, or subcellular components to which they have been provided along with the other genetic material. Genetically marking an entity with TIMs therefore provides a means to monitor the reproduction of the entity. This can be useful, for example, in tracking the location and identity of genetically modified plants and animals and their products. However, those of skill in the art will recognize that an entity need not be capable or reproduction, or need not reproduce, in order to be usefully marked and identified by TIMs. For example, animals that are produced via in vitro fertilization may be marked with TIMs, grown to maturity, and readily identified by detecting the inserted TIM, whether or not the animal is also bred.

Composition of TIMs: General.

TIMs are DNA molecules that contain an identifying central region (ICR) and at least one unique flanking sequence (flanking region, FR) located adjacent to the ICR. In some embodiments, the ICR is bounded by unique FRs, i.e. there are unique FRs adjacent to both the 5′ and 3′ ends of the ICR. By “adjacent to the ICR” we mean immediately contiguous with or in direct continuity by means of covalent linkage. In a preferred embodiment of the present invention, the FRs contain primer binding sites that are unique to the TIMs and are not present in the native DNA of the host. It is thus possible to selectively amplify the ICR of a TIM from within a sample of total DNA from a host by using primers specific for the unique sequences of the FRs. However, those of skill in the art will recognize that even if the DNA sequences that encode the primer binding sites are also found within the host cell native DNA, so long as the native sequences are not located in proximity to one another, PCR amplification from those sites will not occur and will not interfere with the selective amplification of the TIM ICR.

The ICR region itself may or may not be present in the native DNA of the host. Because the presence of a TIM is, in a preferred embodiment, detected by amplification of the ICR by PCR, and because the ICR is selectively amplified (the primer binding sites are unique) the presence of sequences like those of the ICR elsewhere in the host DNA does not interfere with detection of the TIM because those sequences will not be amplified during PCR.

The coupling of an ICR with its unique FRs provides a wealth of possibilities with respect to conferring a unique genetic marker on an entity. The uniqueness is provided at two levels. First, the primer binding sites within the FRs are unique to the entity. Secondly, the ICRs that are detected are highly variable.

Many versatile configurations and combinations of FR sequences and ICRs (discussed below) may be designed in order to render the recipient of a TIM genetically identifiable. “Families” of TIMs may be developed which show the interrelatedness of the various recipients of the TIMs that form such a family. Distinct configurations and combinations may be used to signify any of a wide variety of characteristics of the TIM recipient, including but not limited to: the identity of the developer (e.g. the TIM is used as a company “brand” or “genetic signature”), the type of entity that is being marked (e.g. corn, an immortalized cancer cell line, clonally related livestock, etc.), a specific type of genetic modification (e.g. resistance to a pesticide, ability to produce a non-native protein, etc.), hybrids designed for growth in a specific locale, geographical origin or destination (e.g. varieties produced at a particular plant, or sold in a particular region) and the like. “Families” of TIMs may be developed so that, for example, a single unique pair of flanking sequences is utilized to mark all the genetically modified varieties of corn developed by a company, and a second unique pair of flanking sequences may be utilized to mark all genetically modified varieties of soybeans. Then, within the group of GM corn each variety possessing a different genetic modification (e.g. pest resistance, pesticide resistance, or each hybrid strain, etc.) could be marked with a unique ICR. Likewise, within the group of GM soybeans, each variety possessing a different genetic modification or each hybrid strain may also be marked with a unique ICR. Other ICRs, such as one for resistance to a particular herbicide could be made identical in all groups, regardless of the identity of the unique primers used to amplify the ICR. Alternatively, each individual variety of a GM crop (e.g. a variety that is resistant to a specific pesticide) may have its own unique pair of flanking sequences, and the composition of the corresponding ICR may be altered each year so that the year of introduction of a variety can be monitored. A second TIM could be transected into the hybrid germplasm to identify it prior to introduction of a GMO trait, e.g. pesticide resistance. Because of the large number of ACGT-based sequences that make up the potential pool of flanking sequences and ICRs, and the almost limitless potential for arrangements of primers and ICRs, ample variety exists for providing unique genetic markers for any characteristic or pattern of characteristics that one desires to mark and monitor.

In general, more than one TIM may be inserted into an entity. Its ICR would vary from hybrid to hybrid, thereby identifying different hybrids carrying the same gene trait.

Composition of TIMs: the primers and primer binding sites

The artificial primer binding sites are designed in the following manner: random primer sequences are generated using software designed for this purpose, for example that found at the “Random Sequence Generation” page of the web site “www4.fallingrain.com”. Such random sequence generation programs are, in general, based on statistical methods of random number generation such as that outlined by statistical texts (for example, see Snedecor and Cochran, 1989). Primers may also be designed from the DNA of organisms not closely related to the organism of intended use.

Software (e.g. Primer 3 software from the Whithead MIT genome Center website, or DNASIS software, Hitachi) can be used to screen designated areas of random nucleotide sequence for possible primer binding sites. One criteria for selecting appropriate primers will be that the Tm for the binding of the primer to its site must be within a useful range for PCR amplification, e.g. from 50-70° C., and preferably from 58 to 66° C. Those of skill in the art will recognize that Tm is dependent on primer lengths and GC content, varying according to the formula Tm=81.5+16.6×log[Na+]41×(#G +#C)/length−500/length, where [Na+]=0.1 M.

Thus, another criterion for evaluation is GC content of the primer. In general, the GC to AT ratio should be in the range of 20-80% in order to optimize strength of hybridization and limit hairpin formation. In a preferred embodiment of the present invention, the GC to AT ratio is from 36-57%. The length of the primers must also be taken into consideration. Primer lengths can, in general, range from about 13 nts to about 35 nts, and will preferably range from about at least 18 nts to insure specificity of amplification, to a maximum of about 30 nts to limit the potential for primer dimer formation, especially during multiplex PCR.

Further, the Tms for each for the two primers of a primer pair must be compatible i.e. both must denature at approximately the same temperature (generally within ≦5° C. of each other, and preferably within 3° C. of each other) thereby allowing for efficient annealing of each primer during PCR. If multiple TIMs are to be PCR amplified in a single multiplex reaction, the Tms of the primers for each ICR locus to be amplified will preferably be within 5° C. of each other. Without such thermal compatibility, there may be unequal or incomplete amplification during PCR.

Other caveats for primer design include: a maximum 3′ duplex stability of ≧9 is preferable; the allowable 3′ global alignment score of a single primer for self-complementarity is preferably ≦3 nt; and the maximum allowable number of consecutive repeated nucleotides within a primer is preferably ≦5. Software programs such as Operon Technologies OligoToolkit may be employed to calculate the Tm for each primer and to restrict complimentarily between all primer pairs, both within and between the ICR loci to <7 bp. Software such as DNA Star (DNA Star, Inc.) may be used to restrict maximum complimentarity of primer 3′ ends of individual primers both within and between multiplexes to ≦5 nts.

Once a primer pair has been selected as appropriate, the primers are synthesized and screened for non-specific amplification of the target genome. If a primer pair is found to be incapable of inducing PCR amplification of the host genome of a given entity, then the pair may be utilized in that entity. Primers may also be screened for their ability to hybridize to the DNA of the host organism. However, as discussed above, even it there is some sequence homology between a given PCR primer and the host genome, it is very unlikely that both sequences of a pair would be homologous, or that homologous sequences for a given pair of PCR primers would be located close enough together and in the correct orientation to allow for amplification. Thus, the occurrence of some homology between the primers and host DNA does not per se preclude their use in the practice of the present invention, so long as PCR amplification of host DNA does not occur by use of the primers. Further, some primer pairs may be appropriate for use in some strains, organisms, cell lines, and the like, but not in others due to fortuitous homology with host DNA.

Composition of TIMs: the ICR

The ICRs of TIMs may be generally classified as three different types: STR-based, sequence-based, or mixed (i.e. both STR- and sequence-based.) based ICRs: In the STR-based embodiment of the invention, the ICR comprises at least one tandemly repeated sequence. The tandemly repeated sequences are of from about 2-7 base pairs in length, and individual TIMs of this type are distinguishable from one another due to variations in length, resulting from the length of and/or number of individual repeat units that are present.

STR-based TIMs are comprised of commonly naturally occurring or other simple pyrimidine repeats such as aaat, or pyrimidine/purine repeats, such as atag. In order to avoid interference with their PCR amplification, TIMs can be designed to have a low degree of secondary structure by avoiding a predominance of purine nucleotides, preferably ≦57%.

The size of repeat units will be designed to permit easy detection and discrimination from other closely related TIMs by the analytical instrumentation. The least number of repeat units in an STR-based ICR would be one, and the greatest number would be limited by the repeat unit size and the amplification, detection and gene transfer systems. Dinucleotide repeat units may yield relatively large “shadow” PCR amplification products one repeat unit shorter than the true repeat length due to slippage of the DNA polymerase (Murray, 1993), which may interfere with identification of the true repeat length. Therefore, repeat units of 3-5 nt are often preferred for microsatellite based genetic identification, because they yield less prominent shadow band PCR amplification products. Some trimeric repeats may undergo expansion within an organism, possibly due to the formation of unusual secondary structures during replication (Samadashwily, 1997). Trinucleotide repeat expansion may be blocked by interruption within the repeated DNA. Longer microsatellite repeat units of 6 and 7 nucleotides are stable but have the disadvantage of making the TIM longer for a given number of repeats, thereby lessening the amount of polymorphism available from a given DNA fragment length.

To obtain complete PCR amplification and rapid, accurate electrophoretic analysis of microsatellite loci, a upper limit of 400 bp is frequently selected. Thus, if a tetrameric repeat sequence were being employed, one might design a set of 100 TIMs in which all were amplified by the same pair of primers, and in which the ICR ranged in composition from one to 100 tetrameric repeats. A possible use of such a set of TIMs would be to indicate the year of introduction of a crop seed. Obviously, a generous 100-year span of time could be covered by this one simple TIM design.

Further, the ICRs of STR-based TIMs are not necessarily comprised only a single type of regular uniform repeat unit but may instead contain more than one type of repeat unit. For example, an ICR could contain ten four-nt repeats adjacent to ten two-nt repeats. Or the pattern of repeat units could be even more complex. Any useful combination of repeat units or other nucleotides, either singly or in sequence, may be utilized in the practice of the instant invention.

In order to increase their power of discrimination, it is preferable to insert multiple shorter TIMs rather than a single long TIM, since the power of the former increases geometrically, the latter arithmetically during PCR amplification. For example, two TIMs of 25 and 25 repeat units each when measured independently yield 25×25=625 unique marker combinations, whereas a single TIM of up to 50 repeat units yields only 50 unique markers.

Mixed ICRs: In addition to regular tandem repeat sequences, non-repeat nucleotides may also be introduced to cause the amplified fragment lengths from a TIM to vary in length by a value less than a full repeat unit (“mixed” TIMs). For example, 9 tetrameric repeats in an STR-based ICR would generate a PCR fragment that is 36 nts in length. However, the addition of 1, 2, or 3 nucleotides to 9 tetrameric repeats would create fragments of 37, 38, and 39 nt, respectively, i.e. “repeats” of 9.1, 9.2, and 9.3 units, thereby greatly increasing the discriminating power of the TIM. The resolution of the analytical instrumentation will dictate whether such fractional repeat units can be employed. For example, most sequencing gel electrophoresis systems can reliably discriminate DNA fragments differing by 2 bp; time of flight mass spectrometry can ordinarily discriminate a difference of 1 bp.

Sequence-based ICRs: The ICRs of sequence-based TIMs do not contain tandemly repeated sequences. Instead, the ICRs of sequence-based TIMs vary from each other according to primary sequence and/or length. In either case, they are thus distinguishable from one another by sequence sensitive methods such as DNA sequencing or chip hybridization. In the case of hybridization based detection instruments, the length of complementary DNA must be sufficient to provide enough binding energy for specific hybridization (Wallace, 1997). Any variation in ICR sequence detectable by an analytical instrument would serve to discriminate different cells or organisms. If the length is also varied, then sequence-based TIMs are also distinguishable from one another by analytical techniques that measure length, such as those employed for STR-based TIMs. The sequences that make up the ICR of a sequence-based TIM may or may not be homologous to native DNA sequences of the host entity.

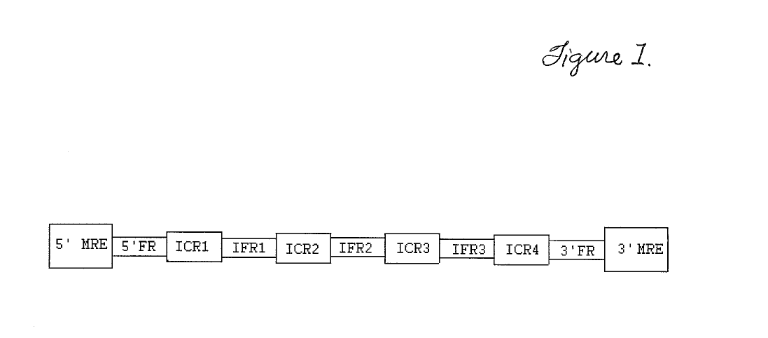

Other variations: TIMs may also contain restriction enzyme sites, for example, to facilitate cloning or analysis. Restriction endonuclease sites may be designed into the sequence so as to bracket the flanking primer binding sites. FIG. 1 illustrates a TIM in which multiple restriction enzyme cutting sites (MRE) have been incorporated at the extreme 5′ and 3′ ends of the TIM. Digestion of the synthetic TIM fragment with an appropriate restriction endonuclease would then allow insertion of the restricted fragment into a cloning site of an appropriate vector such as a gene transfer vector. In addition, TIMs may contain strategically placed restriction enzyme sites to allow for their excision from or joining to each other. Several covalently joined TIMs can then be inserted into a host organism. Further, such strategically placed restriction sites may be useful for “mixing and matching” the components of TIMs in a cassette fashion, e.g. synthetic ICRs and primer binding sites can be removed from a parent molecule via restriction endonuclease digestion and recombined with each other to form alternative ICRs and primer binding site combinations.

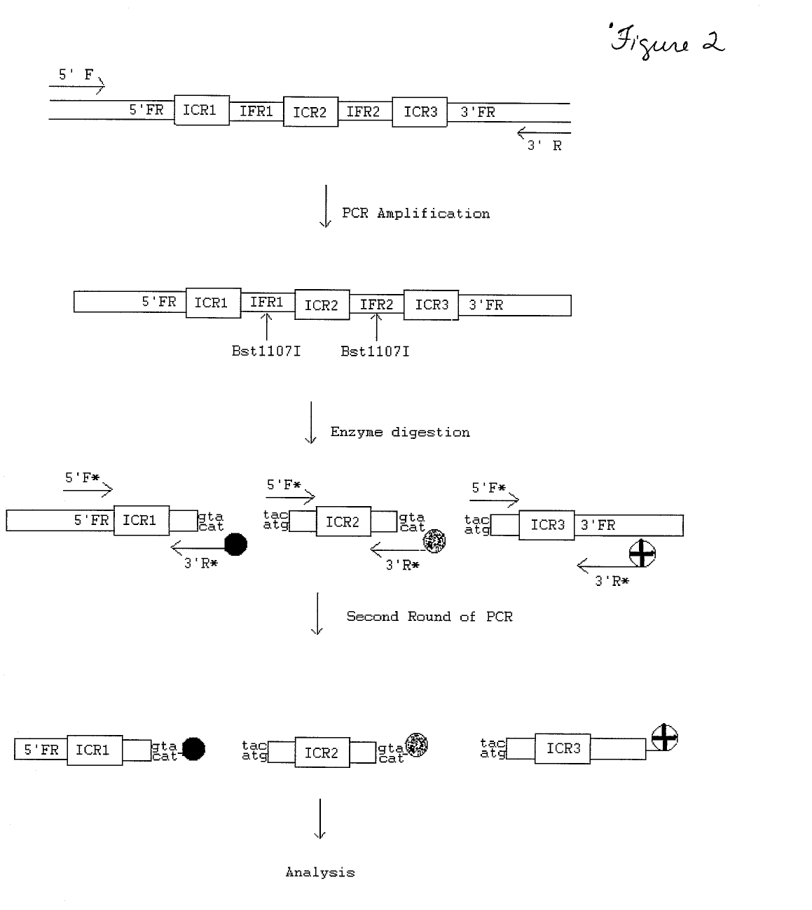

Such restriction sites may also be useful during detection and analysis of TIMs. An Example of the use of the restriction sites in this manner is given in FIG. 2. In this figure, a TIM is depicted which contains three different covalently linked ICRs. The forward and reverse primers for the TIM are indicated. As can be seen, the TIM has been designed to contain restriction enzyme sites for Bst 1107 I between the ICRs, and restriction enzyme digestion of the PCR amplicon is used to facilitate the overall process of detection of the different ICRs by, for example, electrophoresis or chip hybridization. A more detailed explanation of FIG. 2 is given below in the section covering the detection of TIMs.

In addition, TIMs may also contain differential primer sites, i.e. more than one primer pair may be designed to amplify the ICR from the same flanking primer binding sites. For example, the primers may differ in length and position of binding to a binding site. If a primer binds at the innermost nts of the site but not at the nts farthest from the ICR, the nts farthest from the ICR will not be amplified during PCR amplification and the PCR product will be shorter than when a primer that binds to the entire binding site or to its outermost portion, is utilized. In effect, the portion of the binding site farthest from the ICR fails to become part of the ICR in this instance. This is yet another way to introduce versatility into the genetic marking of entities with TIMs.

When multiple ICRs are incorporated into a TIM, each individual ICR may be flanked by unique FR sequences which contain primer binding sites for PCR amplification of the ICR. FIG. 1 illustrates such a case in which four ICRs (ICR1, ICR2, ICR3 and ICR 4) are incorporated into a single TIM. Each ICR is flanked by two unique FRs, and each pair of FRs can be utilized to amplify the ICR which is flanked. The internal FRs are designated “intermediate flanking regions” (IFRs) and are redundant in that they contain binding sites for both reverse and forward primers of the ICRs which precede and follow them, respectively. This case is also illustrative of the need to coordinate the binding parameters of all primers used during PCR amplification of such a TIM to insure that all ICRs are equally and completely amplified. Alternatively, such intermediate flanking regions could also contain restriction sites to permit separation of the individual ICRs by restriction endonuclease digestion prior to PCR, or to permit separation of the individual ICRs by digestion after PCR amplification but before detection.

Alternatively, one or more reverse (3′) primers or forward (5′) primers could be identical in sequence to other reverse or forward primers, respectively. In the case where the PCR products are labeled with a fluorophore covalently attached to the 5′ primer for subsequent electrophoretic analysis, PCR products with the same fluorophore label on different TIMs would have to be non-overlapping in fragment length for discrimination by electrophoresis. Otherwise, they would have to be discriminated from each other through differential fluorophore labeling by means of distinctive 5′ primers.

Synthesis and Assembly of TIMs

TIMs may be wholly or partially synthesized chemically or by recombinant DNA technology including PCR amplification. TIM DNA can be synthesized de novo using any of a number of procedures well known in the art. For example, the β-cyanoethyl phosphoramidite method (Beuacage and Caruthers, 1981); nucleoside H-phosphonate method (Garegg et al, 1986; Gaffney et al., 1988). These chemistries can be performed by a variety of automated oligonucletide synthesizers available in the market. The TIM may be, for example, synthesized in totality and PCR amplified for production of larger quantities.

In order to facilitate storage and manipulation, or to facilitate transfer into the entity which is to be marked by the TIM, the TIM DNA may be inserted into an appropriate vector. Those of skill in the art are familiar with the many types of vectors that are available for such purposes, including but not limited to plasmids, cosmids, viral-based vectors, and the like. The TIM DNA may be inserted into any appropriate vector so long as the integrity of the TIM is not disrupted.

Providing TIMs to the Host

The method of the instant invention comprises inserting into the entity to be genetically marked at least one heritable transgenic identification marker (TIM). When TIMs are inserted into a host, a single TIM or a plurality of TIMS may be inserted. If a plurality of TIMs are inserted, they may or may not be covalently linked to each other. Further, TIMs may be inserted independently from any other element that is also being used to genetically modify the host, or the TIM or TIMs may be covalently linked to another element that is being used to further genetically modify the entity. For example, a TIM may be housed in a vector immediately adjacent to DNA sequences that encode a gene of interest that is being used to genetically modify an organism. Thus, when the vector is introduced into the germ cell of the organism, the TIM will be inserted concomitantly. If the DNA encoding the gene of interest is to be stably integrated into the genome of the host organism, the sequences responsible for integration can be placed so as to flank both the gene of interest and the TIM as a unit. The TIM will then be located adjacent to the gene of interest in the host genome.

Alternatively, the TIM may be introduced into the host on a DNA vector that is separate from that which contains the gene of interest. Insertion may take place before, after or concomitant with insertion of the gene of interest. What is required is that the TIM be inserted into the germ cell line at a time and in a manner that allows reproduction of the TIM as the organism matures. In this way, the mature organism will contain at least one copy of the TIM in most or all cells. Once inserted into a host, the heritable TIM may be integrated into the genome of the entity or maintained extrachromosomally.

Those of skill in the art will recognize that the method of inserting a TIM into a host cell will vary depending on the type and characteristics of the host cell, and that many protocols exist and are used routinely in order to insert DNA into host cells. Bacteria, single cells, and algae: The insertion of TIM DNA into entities such as bacteria, yeast, single-celled organisms, cultured cell lines, and algae may be carried out by techniques that are well-known to those of skill in the art and include but are not limited to, for example, lipid mediated transfection, electroporation, various chemical means, by direct injection of the DNA, or by viral-mediated trasfection. Such techniques are utilized on a routine basis and may be used to insert TIM DNA into a host cell or organism. The TIM DNA may be introduced alone, or in combination with other DNA. The TIM DNA may be covalently attached to DNA encoding, for example, a gene of interest. Alternatively, the TIM DNA may be on a separate DNA molecule.

Plants: The introduction of TIMs into plants as genetic markers can be accomplished by means that are well known to those of skill in the art. For example, dicotyledon plants such as soybean, squash, tobacco (Lin et al. 1995), and tomatoes can be transformed by Agrobacterium-mediated bacterial conjugation. (Miesfeld, 1999, and references therein). In this method, special laboratory strains of the soil bacterium Agrobacterium are used as a means to transfer DNA material directly from a recombinant bacterial plasmid into the host cell. DNA transferred by this method is stably integrated into the genome of the recipient plant cells, and plant regeneration in the presence of a selective marker (e.g. antibiotic resistance) produces transgenic plants.

Alternatively, for monocotyledon plants, such as rice (Lin and Assad-Garcia, 1996), corn, and wheat which may not be susceptible to Agrobacterium-mediated bacterial conjugation, TIMs may be inserted by such techniques as microinjection, electroporation or chemical transformation of plant cell protoplasts (Paredes-Lopez, 1999 and references therein), or particle bombardment using biolistic devices (Miesfeld, 1999; Paredes-Lopez, 1999; and references therein). Monocotyledon crop plants have now been increasingly transformed with Agrobacterium (Hiei, 1997) as well.

Insects: TIM DNA may be inserted into insect species. For Drosophila melanogaster, germ-line DNA integration is accomplished via P element transformation.

Multicellular animals: DNA transfer into embryonic mammalian cells via microinjection, retroviral infection, transfection with a lipid-type compound such as LIPOFECTAMINE™ (lipofection) or by chemical or electrical means (such as electroporation);

For mammalian animals (e.g. mice, goats, cattle, and the like) the transfer of TIM DNA may be accomplished with known techniques for transgenesis. One such technique is the microinjection of male pronuclei of fertilized eggs with the lineraized DNA, followed by implantation into a host female. Similarly, TIM DNA may be introduced into an organism during a nuclear transfer procedure [in which DNA from a donor cell (e.g. a fetal cell) is inserted via microinjection into an enucleated egg cell, or by the fusion (e.g. by electroporation) of a donor cell to an enucleated egg cell], in which the TIM is first introduced into the donor cell via standard DNA co-transfection (e.g. lipid mediated) (Miesfeld, 1999).

Detection of TIMs.

Cells that contain TIMs can be identified by selective amplification of the TIM via, for example, polymerase chain reaction followed by detection of the amplification products (amplicons). In order to carry out a PCR reaction, DNA of the host must first be isolated. Methods for the isolation of DNA from bacteria, cultured cell lines, mammalian cells, insect cells, and the like are well known to those of skill in the art and are relatively straightforward.

However, the isolation of DNA from plant cells may require special attention. Purifying DNA from some plant species can be difficult due to the presence of polyphenolic compounds and polysaccharides which have solubility properties similar to those of DNA and which can interfere with the PCR reaction. As a result, various techniques have bee developed for the isolation of DNA from plant cells. Such techniques include the MasterPure™ Plant Leaf DNA Purification Kit (Epicentre Technologies) which was shown to be effective for the isolation of DNA from late season grape cultivar leaves. The DNA was of sufficient quality to allow PCR amplification of several microsatellite loci (Hoffinan and Moan, 1999), a process which would be TIMilar to that of amplifying the ICRs of a TIM. Alternatively, the use of FTA paper has also been applied to isolation and successful PCR amplification of DNA from diverse species of plants, including Arabidopsis, cannabis, cassava, coca, corn, orchid, papaya, petunia, poppy, potato, rice, soybean, sugarbeet, sugarcane, tobacco and tomato (Lin et al., 2000). The cetyltrimethylammoniumn bromide (CTAB) method is widely used for a variety of leaf tissues (Doyle and Doyle, 1987). High quality DNA may be isolated from the economically important crop plant barley (Shaghai-Maroof, 1984; Sharp et al. 1988). DNA isolation procedures for the carrot have also been oprimized (Boiteux et al., 1999). DNA has successfully been isolated from certain recalcitrant species in the plant families sonneratiaceae, rhizophoracea, myrsinaceae, verenaceae, convolvulaceae, and zingiberaceae by a modified CTAB protocol which utilizes a silica matrix (Huang et al., 2000). Wagner at al. (1987) developed a technique for DNA preparation from lodgepole and jack pines, and this method was adapted by Bryne et al. (1999) to successfully isolate high quality DNA from eucalyptus. The incorporation of sodium sulfite into the method of Wagner has been shown to further stabilize DNA extracted from a number of Acacia species (Byrne et al. 2001). DNA has been successfully isolated from economically important grape cultivars (Bowers et al., 1993; Thomas and Scott, 1993).

Once DNA has been successfully isolated from the host entity, PCR can be carried out by methods which are well-known to those of skill in the art.

The method of detection of the PCR amplicons will vary according to the original design of the ICRs to be detected. In general, ICRs which differ in length (which may be STR-based, sequence- based or mixed) may be detected by any technique that is sensitive to DNA fragments of differing lengths, e.g. slab gel electrophoresis, capillary electrophoresis, mass spectrometry. All ICRs may be detectable by methods sensitive to sequence, for example direct sequencing of amplicons, hybridization based assays (e.g. chip hybridization assays), and the like. In the case of hybridization based detection technology, the length of complementary DNA must be sufficient to provide enough bonding energy for specific hybridization. Those of skill in the art are well acquainted with such methodology because such techniques are utilized on a routine basis for the analysis of naturally occurring genetic variability in a wide variety of DNA-based life forms. Typical well-known analyses include microsatellite analysis for forensic identification in humans and other species. In plants, some analyses are random-amplified polymorphic DNA (RAPD), amplified fragment length polymorphism (AFLP), and microsatellite or simple sequence repeats (SSR) analyses.

If PCR amplicons are to be analyzed by electrophoresis, differential labeling of the amplicons may be necessary. For example, PCR products compiled from TIMs containing multiple ICRs which overlap in length, but which have distinctive flanking regions and are thus amplified by different primer pairs, can be distinguished by differential labeling of each primer pair.

FIG. 2 illustrates another approach to the analysis of TIM amplicons. FIG. 2 depicts a TIM containing three different covalently linked ICRs, ICR1, ICR2, and ICR3. The entire TIM is amplified by PCR using the indicated forward and reverse primers (5′F and 3′R, respectively) to produce an amplicon containing all three ICRs. This PCR product can be detected on an agarose slab gel. The TIM is designed so that blunt-end cutting restriction enzyme sites (e.g. Bst1 107 I as depicted in FIG. 2, the cutting sequence of which is 5′gtatac3′) are incorporated into the TIM between ICR1 and ICR2 and between ICR2 and ICR3. Digestion of the amplicon with Bst1 107 I results in separation of the ICRs. The digested fragments then undergo a second round of PCR amplification with differentially labeled forward or reverse PCR primers represented by 5′F* and 3′R*, respectively. In FIG. 2, the 3′R* primers are depicted as labeled, but the label could also be on the 5′F primers. The differential labels (e.g. 5-FAM, ROX, TAMARA, or JOE) are incorporated into the PCR amplicons. The resulting PCR amplicons are thus differentially labeled and can be detected by, for example, electrophoresis, chip hybridization, or fluorescent in situ hybridization (FISH), or by any method that is suitable for the detection of the labels. Alternatively, a sequencing PCR strategy can be utilized in which only one labeled primer (either forward or reverse) is needed for each digested ICR. This is possible because there are sufficient templates available from the first round of PCR amplification. I would also be possible to analyze the digestion fragments prior to the second round of PCR amplification via a mass sensitive technique such as mass spectrometry.

The following examples are intended to illustrate various embodiments of the instant invention. However, they should not be construed so as to limit the scope of the invention in any way.

### EXAMPLES

### Example 1. Design and Synthesis of TIM-1 and TIM-2 genetic markers.

Two pairs of PCR primers, one to amplify TIM-1 and the other to amplify TIM-2 are developed from 18-25 nt sequences generated by a random sequence software program. Those with melting temperatures of 58-66° C., a GC content of 36 to 57%, a maximum 3′ duplex stability of ≧9, a 3′ global alignment score for self-complementarity of ≦3 and a maximum number of the same consecutive repeated nucleotide of ≦5 are studied pairwise for compatibility with one another during PCR. Initially, primers with a Tm within 3° C. of each other are matched. These pairs are further selected to have a nucleotide complementarity of <7 and a maximum 3′ complementarity of ≦5 nt. PCR primers thus selected are then screened for amplification of PCR products from DNA of the organism intended to receive their TIM insert.

Once primer pairs for TIM-1 and TIM-2 amplification are selected, their design is incorporated into the 5′ and 3′ flanking regions (FR) of their TIM designs. In this example, the TIMs are located on separate DNA molecules. The forward TIM primer amplifies the TIM antisense strand and its sequence therefore forms the TIM 5′FR. The reverse TIM primer amplifies the TIM sense strand and therefore its complementary sequence forms the TIM 3′ FR. One primer is labeled with a fluorophore at its 5′ end to allow fluorescent detection of the PCR amplified TIM upon analytical electrophoresis. TIM-1 and TIM-2 ICRs are designed to contain 5 and 6 tetranucleotide repeat units, respectively, with the sequence “atag” or TIMilar sequences. The entire TIM construct, including 2 FR and ICR, are synthesized with an automated DNA synthesizer.

### Example 2. Cloning of TIM-1 and TIM-2 markers into vectors

Appropriate vectors (e.g. Agrobacterium T-DNA transformation vectors) containing two selection markers (e.g. antibiotic resistance such as kanamycin or hygromycin resistance) one of which is operative in bacteria and one of which is operative in plants, are employed for the transformation of plant cells. A reporter gene (e.g. green fluorescent protein) may also be included. Thus, different TIM constructs can be selected and identified in both bacteria and transgenic plants using linked markers independent of the TIM sequences if desired.

Adapter-primers containing the appropriate restriction sites are utilized to clone the TIM-1 and TIM-2 markers into appropriate (e.g.T-DNA) vectors. First, the TIM DNA is amplified with the adapter primers, the resulting fragments are gel purified, digested with suitable restriction enzymes (e.g. EcoR1, HindIII, and the like) and ligated into the corresponding sites of the vector polylinker region. The ligation products are used to transform an appropriate bacterial strain (e.g. E. coli JM109). Recombinant colonies are selected under conditions that allow for the selection marker to be active (e.g. in the presence of the appropriate antibiotic) and confirmed by PCR and sequencing.

### Example 3. Transformation of Agrobacterium tumefaciens with TIM vectors

Recombinant E. coli containing the TIM vector constructs is grown in culture, and plasmid DNA is isolated and purified. Purified plasmid DNA is used for transformation of a suitable strain of Agrobacterium tumefaciens using, for example, electroporation. A. tumefaciens transformants will be selected on an appropriate antibiotic, and confirmed by PCR analysis.

### Example 4. Transformation of plant cells with Agrobacterium strains containing TIM constructs

Plant cells from a suitable plant of interest (e.g. Arabidopsis thaliana) are transformed using an appropriate method (e.g. floral-dip methology). The plant of interest is grown to a suitable stage (e.g. early flowering stage). Agrobacterium strains containing TIM constructs are grown to late-log phase, pelleted, and resuspended. Plants at the suitable stage of development (e.g. late flowering if Arabidopsis plants are used) are exposed to the TIM constructs (e.g. by dipping the plants into the Agrobacterium solution), allowed to recover for a brief interval (e.g. 24 hours), then allowed to mature and set seed normally. Plants are allowed to dessicate, seeds are harvested and cleaned, and stored dessicated.

### Example 5. Selection of transformants (Ti) generation

The above procedure results in about 1% transformation efficiency (e.g. about 1 transformed seed/100 harvested seeds). Harvested seeds are sterilized with bleach, resuspended and plated on appropriate media containing a selection agent (e.g. an antibiotic). Seedlings surviving drug selection are transformants. Transformation is confirmed by transferring surviving seedlings to soil, isolating single rosette leaves and assaying for the presence and expression of the linked reporter gene, if present. DNA isolated from the transformants is used in assays of varietal identification using TIM-1 or TIM-2 marker identification.

### Example 6. Production of segregating populations of plants

T1 plants are self-pollinated and used in crosses to set up segregating populations of plants. A T2 population (population #1) is produced by allowing the T1 plants to self-pollinate normally. This population segregates 1:2:1 for genotypes TIM/TIM : TIM/+:+/+ (where “+” designates a wildtype plant lacking the TIM marker transgene). A second, mixed-T2 population (population #2) is produced by reciprocally crossing T1 lines containing the TIM-1 marker with those containing the TIM-2 marker. Population #2 segregates 1:1:1:1 for genotypes TIM-1/TIM-2:TIM-1/+:TIM-2/+:+/+. Populations #3 and #3A mimic an introgression approach used in transgenic crop production, by crossing T1 transformants to wild-type non-transformed plants. In the case of TIM-1, crossing a T1 (TIM-1) plant to an untransformed plant produces a population segregating 1:1 for genotypes TIM-1/+:+/+. Appropriate crosses are made to individual plants, and plants are allowed to mature.

### Example 7. Screening segregating populations for TIM markers

Seeds from segregated populations are planted and held at 4° C. for one week to synchronize seed germination, then transferred to growth chambers. Single rosette leaves are harvested from individual plants and used for DNA extractions. Varietal identification is carried out by PCR with the primers amplifying the TIM-1 and TIM-2 markers, followed by analysis with a suitable method such as electrophoresis, mass spectrometry, or chip hybridization.

If desired, randomly-selected plants scoring for the presence or absence of the TIM markers are tested for the presence (or absence) and expression of the linked reporter gene (if present).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Boiteux, L. S., Fonseca, M. E. N. and Simon, P. W. J. Amer. Soc. Hort. Sci. 1999, 124(1):32-38.

Bowers, J. E., Bandman, E. B., and Meredith, C. P. Am. J. Enol. Vitic. 1993, 44: 266-274.

Bowling, A. T., et al., Validation of microsatellite markers for routine horse parentage testing. Animal Genetics 1997; 28(4):247-252.

Bryne, M., Macdonald, B. and Coates, D. Mol. Ecol. 1999, 8:1789-1796.

Bryne, M. et al. BioTechniques, 2001, 30: 742-743.

Doyle, J. J. and Doyle, J. L. Phytochemical Bulletin 1987, 19:11-15.

Gao, A-G., et al., Fungal pathogen protection in potato by expression of a plant defensin peptide. Nature Biotechnology 2000; 18:1307-1310.

Goldman, K. A. Science 2000, 290:457-459.

Goldstein, D. B. and Pollock, D. D. J. Hered. 1997, 88: 335-342.

Hauge, X. Y. and Litt, M. A. Hum. Mol. Genetics 1993, 2:411-415.

Hiei, Y et al. Plant Mol. Biol. 1997, 35:205-218.

Hoffman L and Moan E. Epicentre Forum, 1999, 6:1.

Huang, J. Ge, X. and Sun, M. BioTechniques 2000, 28:432-434.

Kishore, G. M., et al., Biotechnology: Enhancing human nutrition in developing and developed worlds. Proc Natl Acad Sci USA 1999; 96:5968-5972.

Lin, J. -J. and Assad-Garcia, In Vitro 1996; 32:35A-36A.

Lin, J. -J., Assad-Garcia, N. and Kuo, J. Plant Science 1995; 109:171-177.

Lin J. -J., Fleming, R., Kuo, J., Matthews, B. F. and Saunders, J. A. BioTechniques, 2000; 28:346-350.

Mahtani, M. M., et al., A polymorphic X-linked tetranucleotide repeat locus displaying a high rate of new mutation: implications for mechanisms of mutation at short tandem repeat loci. Human Molecular Genetics 1993; 2(4):431-437.

Mann, C. C., Crop scientists seek a new revolution. Science 1999; 283:310-314.

Mann, C. C., Genetic engineers aim to soup up crop photosynthesis. Science 1999; 283:314-316.

Miesfeld, R. L. Applied Molecular Genetics, 1999; Wiley-Liss, publisher, pp. 205-235.

Moffat, A. S., Science 2000; 290:253-254.

Murray, V, Monchawin, C and England, P R. Nucleic Acids Research 1993, 21-2395-2398.

Paredes-Lopez, ed. Molecular Biotechnology for Plant Food Production, Technomic Publishing, Inc. 1999; 83-86.

Primmer, C. R., et al., Nature Genetics 1996; 13:391-393.

Samadashwily, G M, Raca, G and Mirkin, S M. Nature Genetics 1997, 17:298-304.

Sanchez-Escribano, E. M., Genome 1999; 42(1):87-93.

Schlotterer, C and Tautz, D. 1992, Nucleic Acids Research, 20:211-216.

Schug, M. D., Wetterstrand, K. A., Gaudette, M. s, Lim R. H., Hutter, C. H. and Aquandro, C. F. 1998, Mol. Ecol. 7:57-69.

Shaghai-Maroof, Proc. Natl. Acad. Sci. 1984, 81: 8014-8018.

Sharp, P J, Kreiss, M, Shewry, P, Gale M D Theor Appl Genet 1988, 75: 286-290.

Snedecor, G. W. and Cochran, W. G. Statistical Methods, Iowa State University Press, 1989.

Strand M., Prolla, T. A., Liskay, R. M. and Petes, T. M., 1993, Nature (London) 365:274-276,

Swanston, J. S., Journal, 1999; 5(2):103-109.

Tessier, N., et al., Population structure and impact of supportive breeding inferred from mitochondrial and microsatellite DNA analyses in land-locked Atlantic salmon Salmo salar J. Molecular Ecology 1997; 6:735-750.

Thomas M.. R. and Scott, N. S. Theor. Appl. Genet. 1993, 86: 985-990.

Toth, G., et al., Microsatellites in different eukaryotic genomes: Survey and analysis. Genome Research 2000; 10:967-981.

U.S. Food and Drug Administration, Office of Premarket Approval. Foods derived from new plant varieties derived through recombinant DNA technology. (web site location: vm.cfsan.fda.gov) Dec. 1999.

Vasil, I. K. (ed.), Molecular improvement of cereal crops. Kluwer Academic Publishers 1999, Dordrecht, The Netherlands.

Vergunst, A. C., et al., Recombination in the plant genome and its application in biotechnology. Critical Reviews in Plant Sciences 1999; 18(1):1-31.

Vosman, B., et al., Molecular characterization of GATA/GACA microsatellite repeats in tomato. Genome 1997; 40:25-33.

Wagner, D. B., Furnier, G. R., Saghasi-Maroof, M. A., Willimas, S. M., Dancik, B. P. and Allard, R. W. Proc. Natl. Acad. Sci. 1987, 84:2097-2100.

Waldbieser, G. C., et al., Cloning and characterization of microsatellite loci in channel catfish, ictalurus punctatus. Animal Genetics 1997; 28(4):295-298.

Wallace, R. Molecular Medicine Today, 1997, 3:384-389.

Weber, J. L., Informativeness of human (dC-dA)n.(dG-dT)n polymorphisms. Genomics 1990; 7(4):524-530.

Weikard, R., et al., Targeted development of microsatellite markers from the defined region of bovine chromosome 6q21-31. Mammalian Genome 1997; 8:836-840.

Yu, K., Abundance and variation of microsatellite DNA sequences in beans (Phaseolus and Vigna). Genome 1999; 42(1):27-34.

We claim:

1. A method for identifying an entity or progeny of said entity, wherein said entity is receptive to genetic modification and wherein DNA is the heritable genetic material of said entity, comprising,
inserting into an entity at least one heritable transgenic identification marker, wherein said heritable transgenic identification marker comprises an identifying central region and at least one unique flanking sequence located adjacent to said identifying central region; and
detecting said heritable transgenic identification marker in said entity or said progeny wherein said step of detecting serves to identify said entity or said progeny.

2. The method of claim 1 wherein said identifying central region comprises at least one tandemly repeated sequence.

3. The method of claim 2 wherein said identifying central region further comprises additional non-repeating nucleotides.

4. The method of claim 1 wherein said identifying central region comprises a non-tandemly repeated sequence.

5. The method of claim 1 wherein said heritable transgenic identification marker further comprises sites selected from the group consisting of restriction enzyme sites and differential primer sites.

6. The method of claim 1 wherein said step of detecting is carried out by amplification by polymerase chain reaction followed by a technique selected from the group consisting of electrophoresis, mass spectrometry, and hybridization.

7. The method of claim 1 wherein said entity is genetically modified prior to said step of inserting.

8. The method of claim 1 wherein said entity is genetically modified concomitant with said step of inserting.

9. The method of claim 1 wherein said entity is genetically modified after said step of inserting.

10. The method of claim 1 wherein said entity is selected from the group consisting of plants, cells derived from plants, subcellular components derived from plants, animals, cells derived from animals, subcellular components derived from animals, transgenic organisms, transgenic cells, isogenic organisms, isogenic cells, chimeric organisms, chimeric cells, fungi, bacteria, viruses, insects, algae, and protozoa.

11. The method of claim 1 wherein said heritable transgenic identification marker is integrated into the genome of said entity.

12. The method of claim 1 wherein said heritable transgenic identification marker is maintained extrachromasomally within said entity.

13. The method of claim 1 wherein a plurality of said heritable transgenic identification markers are inserted into said entity.

14. The method of claim 13 wherein said plurality of heritable transgenic identification markers are covalently linked.

15. The method of claim 1 wherein said step of inserting is carried out by transfection of said heritable transgenic identification marker into said entity.

16. The method of claim 1 wherein said step of inserting is carried out by transduction of said heritable transgenic identification marker into said entity.

17. A method of identifying a host, comprising,
detecting a transgenic identification marker in said host, wherein said transgenic identification marker comprises an identifying central region and at least one unique flanking sequence located adjacent to said identifying central region.

18. The method of claim 17 wherein said identifying central region comprises at least one tandemly repeated sequence.

19. The method of claim 18 wherein said identifying central region further comprises additional non-repeating nucleotides.

20. The method of claim 17 wherein said identifying central region comprises a non-tandemly repeated sequence.

21. The method of claim 17 wherein said transgenic identification marker further comprises sites selected from the group consisting of restriction enzyme sites and differential primer sites.

22. The method of claim 17 wherein said host is selected from the group consisting of plants, cells derived from plants, subcellular components derived from plants, animals, cells derived from animals, subcellular components derived from animals, transgenic organisms, transgenic cells, isogenic organisms, isogenic cells, chimeric organisms, chimeric cells, fungi, bacteria, viruses, insects, algae, and protozoa.

23. A host stably transfected with a heritable transgenic identification marker wherein said heritable transgenic identification marker comprises an identifying central region and at least one unique flanking sequence located adjacent to said identifying central region.

24. The host of claim 23 wherein said identifying central region comprises at least one tandemly repeated sequence.

25. The host of claim 24 wherein said identifying central region further comprises additional non-repeating nucleotides.

26. The host of claim 23 wherein said identifying central region comprises a non-tandemly repeated sequence.

27. The host of claim 23 wherein said transgenic identification marker further comprises sites selected ftom the group consisting of restriction enzyme sites and differential primer sites.

28. The host of claim 23 wherein said host is selected from the group consisting of plants, cells derived from plants, subcellular components derived ftom plants, animals, cells derived from animals, subcellular components derived from animals, transgenic organisms, transgenic cells, isogenic organisms, isogenic cells, chimeric organisms, chimeric cells, fungi, bacteria, viruses, insects, algae, and protozoa.

29. A host transformed with a transgenic identification marker wherein said transgenic identification marker comprises an identifying central region and at least one unique flanking sequence located adjacent to said identifying central region.

30. A plant transformed with a transgenic identification marker wherein said transgenic identification marker comprises an identifying central region and at least one unique flanking sequence located adjacent to said identifying central region.

31. An animal transformed with a transgenic identification marker wherein said transgenic identification marker comprises an identifying central region and at least one unique flanking sequence located adjacent to said identifying central region.

32. A cell line transformed with a transgenic identification marker wherein said transgenic identification marker comprises an identifying central region and at least one unique flanking sequence located adjacent to said identifying central region.
